(19) United States
(12) Patent Application Publication
(10) Pub. No.: US 2025/0101057 A1
(43) Pub. Date: Mar. 27, 2025

(54) SOLID FORMS

(71) Applicant: Gilead Sciences, Inc., Foster City, CA (US)

(72) Inventor: Bing Shi, Redwood City, CA (US)

(21) Appl. No.: 18/754,320

(22) Filed: Jun. 26, 2024

Related U.S. Application Data
(60) Provisional application No. 63/523,505, filed on Jun. 27, 2023.

(51) Int. Cl. C07H 19/173; A61K 31/7076

(52) U.S. Cl. CPC C07H 19/173; A61K 31/7076

(57) ABSTRACT

The present disclosure relates to crystalline and solvate forms of a nucleoside reverse transcriptase translocation inhibitor (NRTTI), and pharmaceutical compositions thereof, which are useful in the treatment and prevention of a Retroviridae viral infection including an infection caused by the HIV virus.

### CROSS-REFERENCE TO RELATED APPLICATIONS

This application claims the benefit under 35 U.S.C. § 119(e) of U.S. Provisional Application No. 63/523,505, filed on Jun. 27, 2023, which is hereby incorporated by reference in its entirety.

### TECHNICAL FIELD

The present disclosure relates to crystalline and solvate forms of a nucleoside reverse transcriptase translocation inhibitor (NRTTI), and pharmaceutical compositions thereof, for use in the treatment or prevention of a Retroviridae viral infection including an infection caused by the HIV virus.

### BACKGROUND

Positive-single stranded RNA viruses comprising the Retroviridae family include those of the subfamily Orthoretrovirinae and genera Alpharetrovirus, Betaretrovirus, Gammaretrovirus, Deltaretrovirus, Epsilonretrovirus, Spumavirus, and Lentivirus, which cause many human and animal diseases. Among the Lentivirus, HIV-1 infection in humans leads to depletion of T helper cells and immune dysfunction, producing immunodeficiency and vulnerability to opportunistic infections.

One approach to treating HIV-1 infection is by administering NRTTIs. NRTTIs inhibit HIV-1 reverse transcriptase and, because reverse transcriptase function is essential for viral replication and production of viral proteins, NRTTIs can be effective against HIV-1 infection. Curr Opin HIV AIDS. 2018 July; 13(4): 294-299. HIV treatments, however, have historically lead to the emergence of HIV strains that are resistant to current therapies. Expert Opin Emerg Drugs. 2018 June; 23(2): 149-157. Therefore, there is an ongoing need to discover new antiretroviral agents and to develop methods for their preparation and purification as well as prepare improved pharmaceutical formulations of the same. The solid forms of NRTTIs disclosed herein help meet these and other needs.

### SUMMARY

The present disclosure provides, inter aha, a crystalline form of (2R,3S,5R)-5-(6-amino-2-fluoro-9H-purin-9-yl)-2-ethynyl-2-((2-phenylacetoxy)methyl)tetrahydrofuran-3-yl 2-phenylacetate, which is selected from crystalline Form II, crystalline Form III, crystalline Form IV, and crystalline Form V.

The present disclosure further provides a solvate form of (2R,3S,5R)-5-(6-amino-2-fluoro-9H-purin-9-yl)-2-ethynyl-2-((2-phenylacetoxy)methyl)tetrahydrofuran-3-yl 2-phenylacetate.

The present disclosure further provides a pharmaceutical composition comprising a crystalline or solvate form disclosed herein, and at least one pharmaceutically acceptable excipient.

The present disclosure further provides a method of treating or preventing a human immunodeficiency virus (HIV) infection comprising administering a therapeutically effective amount of a crystalline or solvate form disclosed herein, to a subject in need thereof.

The present disclosure further provides a crystalline or solvate form disclosed herein, for use in therapy.

The present disclosure further provides a crystalline or solvate form disclosed herein, for use in a method of treating or preventing a human immunodeficiency virus (HIV) infection, comprising administering a therapeutically effective amount of the a crystalline or solvate form to a subject in need thereof.

### DESCRIPTION OF DRAWINGS

FIG. 1 shows a representative X-Ray powder diffraction (XRPD) pattern of Compound 1, acetone solvate 1.

FIG. 2 shows a representative XRPD pattern of Compound 1, crystalline Form II.

FIG. 3 shows a representative differential scanning calorimetry (DSC) thermogram of Compound 1, crystalline Form II.

FIG. 4 shows a representative thermogravimetric analysis (TGA) thermogram of Compound 1, crystalline Form II.

FIG. 5 shows a representative dynamic vapor sorption (DVS) analysis of Compound 1, crystalline Form II.

FIG. 6 shows representative XRPD patterns of Compound 1, solvate forms. From top to bottom: DCM solvate, acetone solvate 2, methyl ethyl ketone solvate, ethyl acetate solvate, methyl acetate solvate, n-butyl acetate solvate, tetrahydrofuran solvate, 1-butanol solvate, p-dioxane solvate, and heptane solvate.

FIG. 7 shows the asymmetric unit of Compound 1, crystalline Form III.

FIG. 8 shows a representative XRPD pattern of Compound 1, crystalline Form III.

FIG. 9 shows a representative DSC thermogram of Compound 1, crystalline Form III.

FIG. 10 shows a representative TGA thermogram of Compound 1, crystalline Form III.

FIG. 11 shows a representative DVS analysis of Compound 1, crystalline Form III.

FIG. 12 shows a representative XRPD pattern of Compound 1, toluene solvate.

FIG. 13 shows a representative XRPD pattern of Compound 1, crystalline Form IV.

FIG. 14 shows a representative DSC thermogram of Compound 1, crystalline Form IV.

FIG. 15 shows a representative TGA thermogram of Compound 1, crystalline Form IV.

FIG. 16 shows a representative DVS analysis of Compound 1, crystalline Form IV.

FIG. 17 shows a representative XRPD patterns of Compound 1, xylene solvate (top) and DMAc solvate (bottom).

FIG. 18 shows a representative XRPD pattern of Compound 1, crystalline Form V.

FIG. 19 shows a representative DSC thermogram of Compound 1, crystalline Form V.

FIG. 20 shows a representative TGA thermogram of Compound 1, crystalline Form V.

FIG. 21 shows a representative DVS analysis of Compound 1, crystalline Form V.

### DETAILED DESCRIPTION

The present invention relates to new solid forms, which are crystalline forms or solvate forms of (2R,3S,5R)-5-(6-amino-2-fluoro-9H-purin-9-yl)-2-ethynyl-2-((2-phenylacetoxy)methyl)tetrahydrofuran-3-yl 2-phenylacetate (i.e., Compound 1, structure shown below; see e.g. U.S. Publication Nos.: 20220323476A1 and 20220332751A1, the disclosures of each of which are incorporated herein by reference in their entireties).

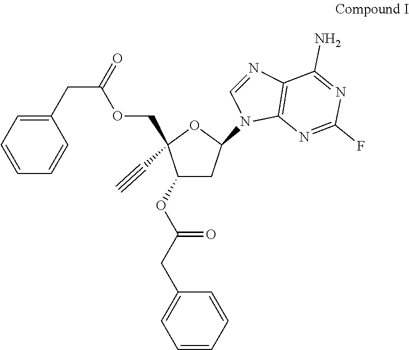 [SMILES: C#C[C@]1(COC(=O)Cc2ccccc2)O[C@@H](n2cnc3c(N)nc(F)nc32)C[C@@H]1OC(=O)Cc1ccccc1]

Compound 1 is a prodrug of 4′-ethynyl-2-fluoro-2′-deoxyadenosine (i.e., islatravir or (2R,3S,5R)-5-(6-amino-2-fluoro-9H-purin-9-yl)-2-ethynyl-2-(hydroxymethyl)tetrahydrofuran-3-ol), a nucleoside reverse transcriptase translocation inhibitor (NRTTI).

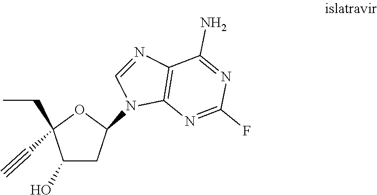 [SMILES: C#C[C@]1(CC)O[C@@H](n2cnc3c(N)nc(F)nc32)C[C@@H]1O]

One skilled in the art understands that a compound structure may be named or identified using commonly recognized nomenclature systems and symbols. By way of example, the compound may be named or identified with common names, systematic or non-systematic names. The nomenclature systems and symbols that are commonly recognized in the art of chemistry including but not limited to Chemical Abstract Service (CAS) and International Union of Pure and Applied Chemistry (IUPAC). Accordingly, the compound structure for Compound 1 provided herein may also be named or identified as (2R,3S,5R)-5-(6-amino-2-fluoro-9H-purin-9-yl)-2-ethynyl-2-((2-phenylacetoxy)methyl)tetrahydrofuran-3-yl 2-phenylacetate.

The present invention provides a crystalline form of (2R,3S,5R)-5-(6-amino-2-fluoro-9H-purin-9-yl)-2-ethynyl-2-((2-phenylacetoxy)methyl)tetrahydrofuran-3-yl 2-phenylacetate, which is selected from crystalline Form II, crystalline Form III, crystalline Form IV, and crystalline Form V.

As used herein, “crystalline form” is meant to refer to a certain lattice configuration of a crystalline substance. Different crystalline forms of the same substance typically have different crystalline lattices (e.g., unit cells) which are attributed to different physical properties that are characteristic of each of the crystalline forms. In some instances, different lattice configurations have different water or solvent content. In some embodiments, the crystalline form provided herein may be substantially anhydrous.

### Compound 1, Crystalline Form II

In some embodiments, the crystalline form provided herein is crystalline Form II.

In some embodiments, the crystalline Form II has at least five XRPD peaks, in terms of 2-theta±0.2°, selected from 5.5°, 9.3°, 10.8°, 14.9°, 18.5°, 19.3°, 23.8°, 24.3°, and 28.4°.

In some embodiments, the crystalline Form II has at least four XRPD peaks, in terms of 2-theta±0.2°, selected from 5.5°, 9.3°, 10.8°, 14.9°, 18.5°, 19.3°, 23.8°, 24.3°, and 28.4°.

In some embodiments, the crystalline Form II has at least three XRPD peaks, in terms of 2-theta±0.2°, selected from 5.5°, 9.3°, 10.8°, 14.9°, 18.5°, 19.3°, 23.8°, 24.3°, and 28.4°.

In some embodiments, the crystalline Form II has at least two XRPD peaks, in terms of 2-theta±0.2°, selected from 5.5°, 9.3°, 10.8°, 14.9°, 18.5°, 19.3°, 23.8°, 24.3°, and 28.4°.

In some embodiments, the crystalline Form II has at least one XRPD peak, in terms of 2-theta±0.2°, selected from 5.5°, 9.3°, 10.8°, 14.9°, 18.5°, 19.3°, 23.8°, 24.3°, and 28.4°.

In some embodiments, the crystalline Form II has XRPD peaks, in terms of 2-theta±0.2°, selected from 5.5°, 9.3°, 10.8°, 14.9°, 18.5°, 19.3°, 23.8°, 24.3°, and 28.4°.

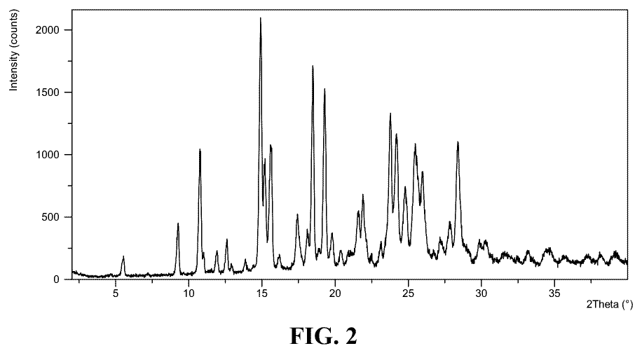

In some embodiments, the crystalline Form II is characterized by an XRPD pattern substantially as shown in FIG. 2.

In some embodiments, the crystalline Form II is characterized by a DSC thermogram having an endothermic transition at about 93° C.

In some embodiments, the crystalline Form II is characterized by a DSC thermogram having an endothermic transition at about 93° C., corresponding to a transition from Compound 1, crystalline Form II to Compound 1, crystalline Form V.

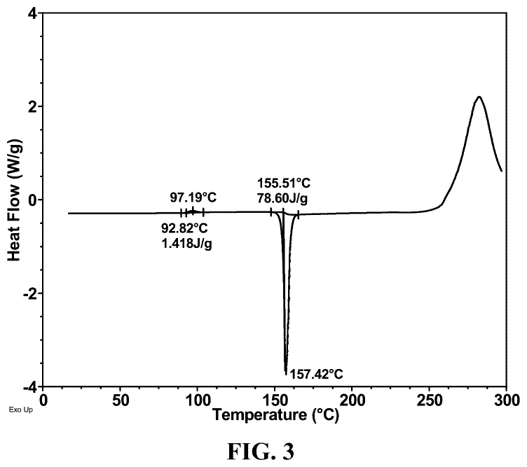

In some embodiments, the crystalline Form II is characterized by a DSC thermogram substantially as shown in FIG. 3.

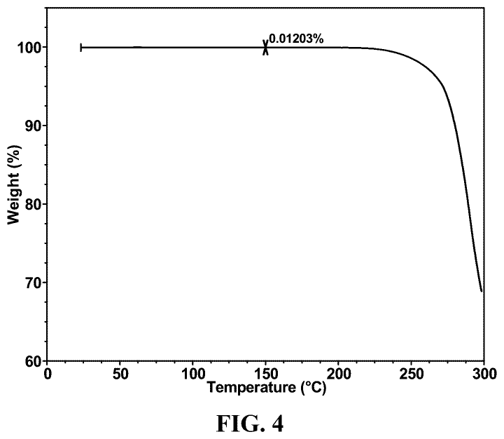

In some embodiments, the crystalline Form II is characterized by a TGA thermogram substantially as shown in FIG. 4.

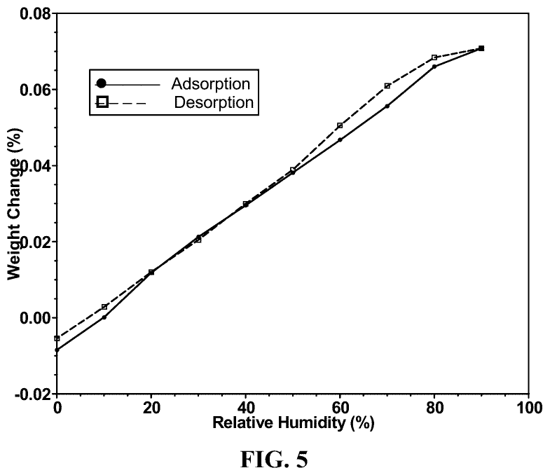

In some embodiments, the crystalline Form II is characterized by a DVS analysis substantially as shown in FIG. 5.

### Compound 1, Crystalline Form III

In some embodiments, the crystalline form provided herein is crystalline Form III.

In some embodiments, the crystalline Form III has at least five XRPD peaks, in terms of 2-theta±0.2°, selected from 7.8°, 11.6°, 13.0°, 14.2°, 16.8°, 21.8°, 25.8°, 26.1°, and 28.0°.

In some embodiments, the crystalline Form III has at least four XRPD peaks, in terms of 2-theta±0.2°, selected from 7.8°, 11.6°, 13.0°, 14.2°, 16.8°, 21.8°, 25.8°, 26.1°, and 28.0°.

In some embodiments, the crystalline Form III has at least three XRPD peaks, in terms of 2-theta±0.2°, selected from 7.8°, 11.6°, 13.0°, 14.2°, 16.8°, 21.8°, 25.8°, 26.1°, and 28.0°.

In some embodiments, the crystalline Form III has at least two XRPD peaks, in terms of 2-theta±0.2°, selected from 7.8°, 11.6°, 13.0°, 14.2°, 16.8°, 21.8°, 25.8°, 26.1°, and 28.0°.

In some embodiments, the crystalline Form III has at least one XRPD peak, in terms of 2-theta±0.2°, selected from 7.8°, 11.6°, 13.0°, 14.2°, 16.8°, 21.8°, 25.8°, 26.1°, and 28.0°.

In some embodiments, the crystalline Form III has XRPD peaks, in terms of 2-theta±0.2°, selected from 7.8°, 11.6°, 13.0°, 14.2°, 16.8°, 21.8°, 25.8°, 26.1°, and 28.0°.

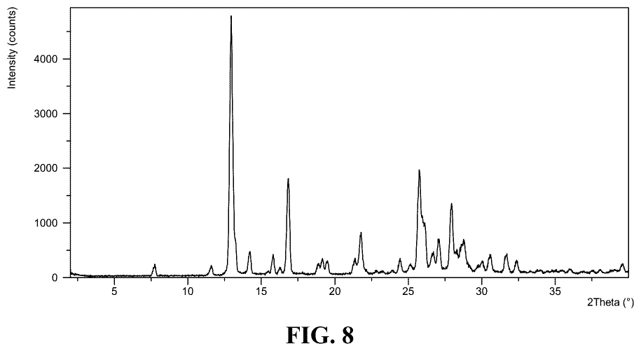

In some embodiments, the crystalline Form III is characterized by an XRPD pattern substantially as shown in FIG. 8.

In some embodiments, the crystalline Form III is characterized by a DSC thermogram having an endothermic transition at about 125° C. In some embodiments, the crystalline Form III is characterized by a DSC thermogram having an endothermic transition at about 125° C. corresponding to a transition from Compound 1, crystalline Form III to Compound 1, crystalline Form V.

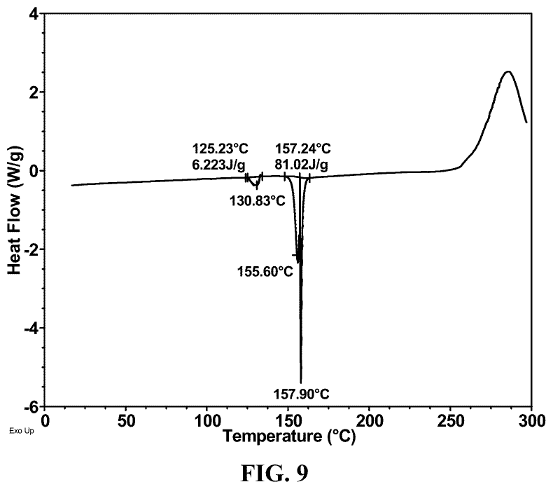

In some embodiments, the crystalline Form III is characterized by a DSC thermogram substantially as shown in FIG. 9.

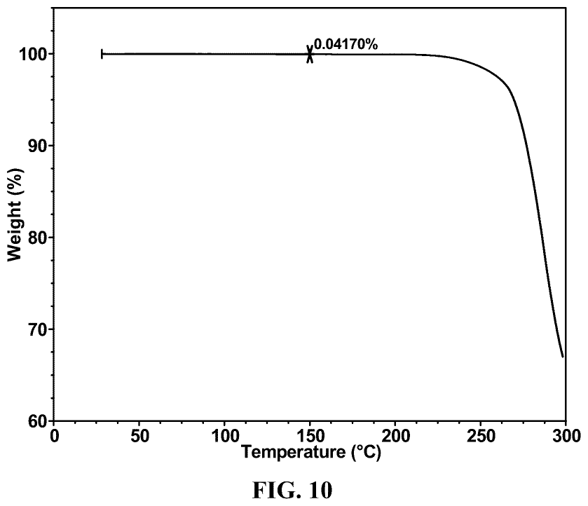

In some embodiments, the crystalline Form III is characterized by a TGA thermogram substantially as shown in FIG. 10.

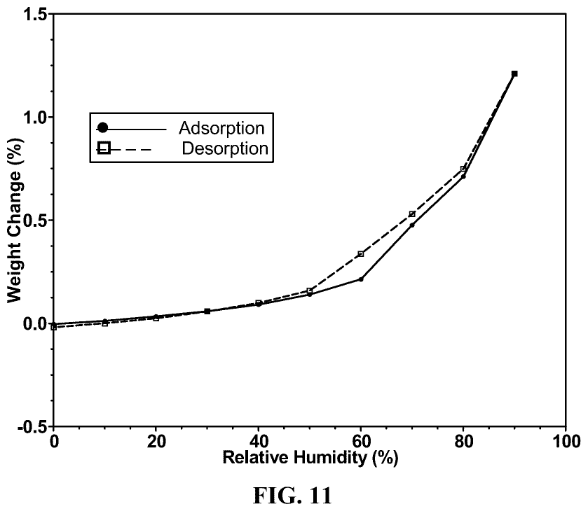

In some embodiments, the crystalline Form III is characterized by a DVS analysis substantially as shown in FIG. 11.

### Compound 1, Crystalline Form IV

In some embodiments, the crystalline form provided herein is crystalline Form IV.

In some embodiments, the crystalline Form IV has at least five XRPD peaks, in terms of 2-theta±0.2°, selected from 8.4°, 12.2°, 12.8°, 14.8°, 15.9°, 18.0°, 24.4°, 24.8°, and 25.8°.

In some embodiments, the crystalline Form IV has at least four XRPD peaks, in terms of 2-theta±0.2°, selected from 8.4°, 12.2°, 12.8°, 14.8°, 15.9°, 18.0°, 24.4°, 24.8°, and 25.8°.

In some embodiments, the crystalline Form IV has at least three XRPD peaks, in terms of 2-theta±0.2°, selected from 8.4°, 12.2°, 12.8°, 14.8°, 15.9°, 18.0°, 24.4°, 24.8°, and 25.8°.

In some embodiments, the crystalline Form IV has at least two XRPD peaks, in terms of 2-theta±0.2°, selected from 8.4°, 12.2°. 12.8°, 14.8°, 15.9°, 18.0°, 24.4°, 24.8°, and 25.8°.

In some embodiments, the crystalline Form IV has at least one XRPD peak, in terms of 2-theta±0.2°, selected from 8.4°, 12.2°, 12.8°, 14.8°, 15.9°, 18.0°, 24.4°, 24.8°, and 25.8°.

In some embodiments, the crystalline Form IV has XRPD peaks, in terms of 2-theta±0.2°, selected from 8.4°, 12.2°, 12.8°, 14.8°. 15.9°, 18.0°, 24.4°, 24.8°, and 25.8°.

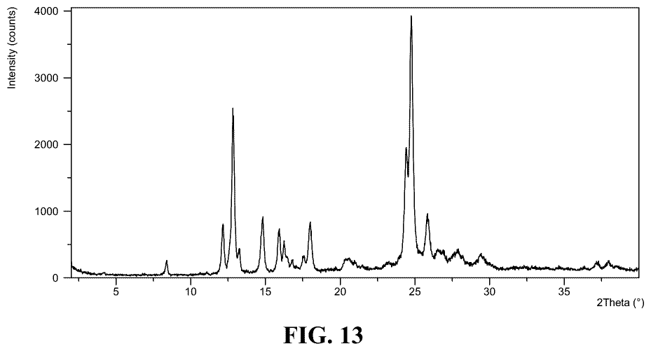

In some embodiments, the crystalline Form IV is characterized by an XRPD pattern substantially as shown in FIG. 13.

In some embodiments, the crystalline Form IV is characterized by a DSC thermogram having an endothermic transition at about 114° C. In some embodiments, the crystalline Form IV is characterized by a DSC thermogram having an endothermic transition at about 114° C., corresponding to a transition from Compound 1, crystalline Form IV to Compound 1, crystalline Form I.

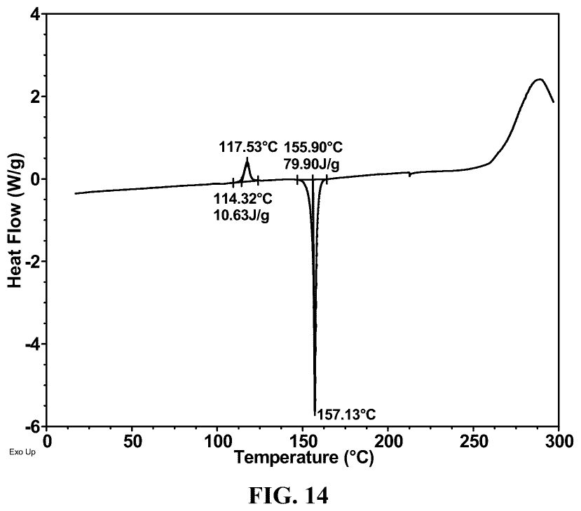

In some embodiments, the crystalline Form IV is characterized by a DSC thermogram substantially as shown in FIG. 14.

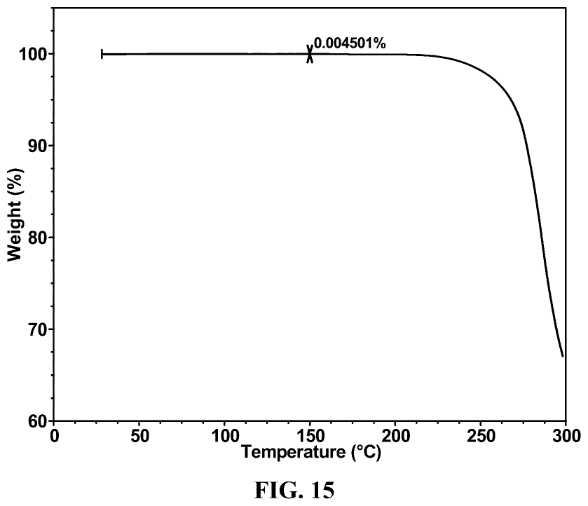

In some embodiments, the crystalline Form IV is characterized by a TGA thermogram substantially as shown in FIG. 15.

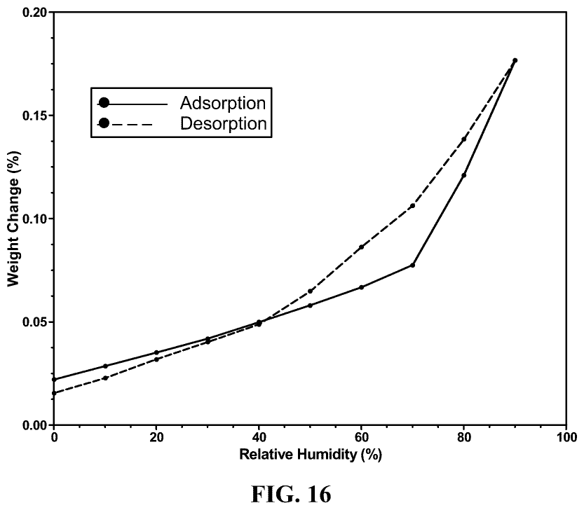

In some embodiments, the crystalline Form IV is characterized by a DVS analysis substantially as shown in FIG. 16.

### Compound 1, Crystalline Form V

In some embodiments, the crystalline form provided herein is crystalline Form V.

In some embodiments, the crystalline Form V has at least five XRPD peaks, in terms of 2-theta±0.2°, selected from 5.4°, 9.0°, 11.2°, 15.10, 15.4°, 18.0°, 19.6°, 20.9°, and 22.30.

In some embodiments, the crystalline Form V has at least four XRPD peaks, in terms of 2-theta±0.2°, selected from 5.4°, 9.0°, 11.2°, 15.1°, 15.4°, 18.0°, 19.60, 20.9°, and 22.3°.

In some embodiments, the crystalline Form V has at least three XRPD peaks, in terms of 2-theta±0.2°, selected from 5.4°, 9.0°, 11.2°, 15.1°, 15.4°, 18.0°, 19.6°, 20.9°, and 22.3°.

In some embodiments, the crystalline Form V has at least two XRPD peaks, in terms of 2-theta±0.2°, selected from 5.4°, 9.0°, 11.2°, 15.1°, 15.4°, 18.0°, 19.6°, 20.9°, and 22.3°.

In some embodiments, the crystalline Form V has at least one XRPD peak, in terms of 2-theta±0.2°, selected from 5.4°, 9.0°, 11.2°, 15.1°, 15.4°, 18.0°, 19.6°, 20.9°, and 22.3°.

In some embodiments, the crystalline Form V has XRPD peaks, in terms of 2-theta 0.2°, selected from 5.4°, 9.0°, 11.2°, 15.1°, 15.4°, 18.0°, 19.6°, 20.9°, and 22.3°.

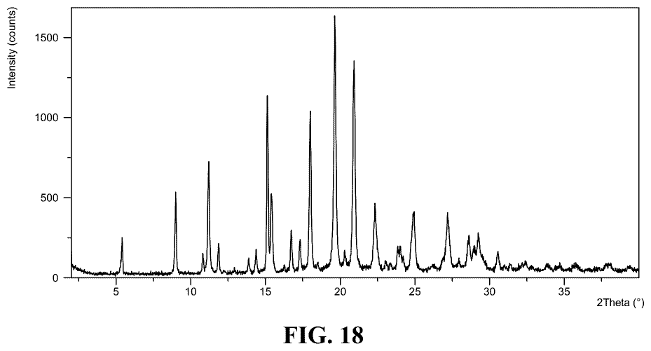

In some embodiments, the crystalline Form V is characterized by an XRPD pattern substantially as shown in FIG. 18.

In some embodiments, the crystalline Form V is characterized by a DSC thermogram having a melting onset of about 156° C.

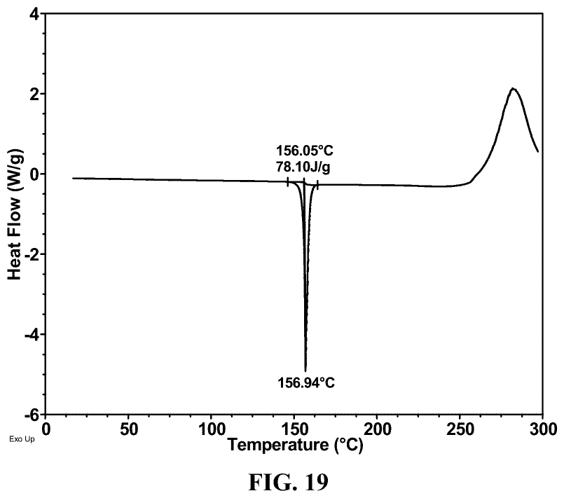

In some embodiments, the crystalline Form V is characterized by a DSC thermogram substantially as shown in FIG. 19.

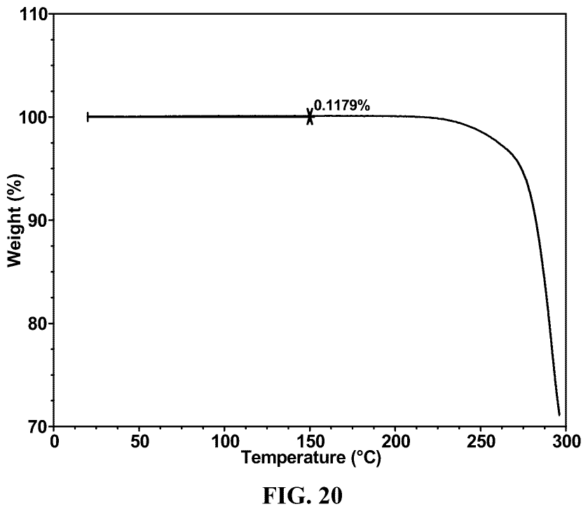

In some embodiments, the crystalline Form V is characterized by a TGA thermogram substantially as shown in FIG. 20.

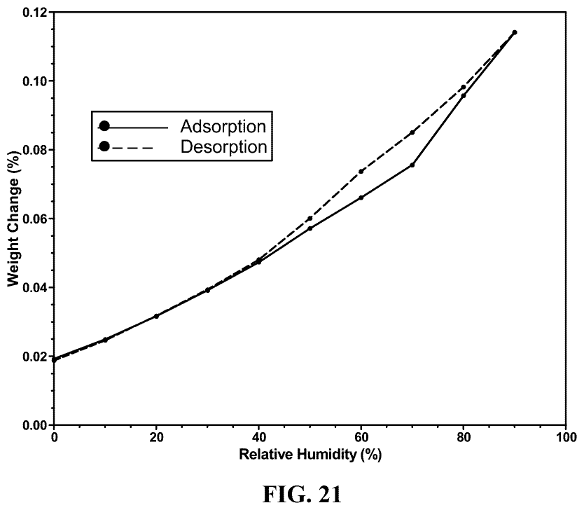

In some embodiments, the crystalline Form V is characterized by a DVS analysis substantially as shown in FIG. 21.

### Compound 1, Solvate Forms

A “solvate” is formed by the interaction of a solvent and a compound. Solvates of the compounds provided herein are also provided. Accordingly, the present invention further provides solvate forms of (2R,3S,5R)-5-(6-amino-2-fluoro-9H-purin-9-yl)-2-ethynyl-2-((2-phenylacetoxy)methyl)tetrahydrofuran-3-yl 2-phenylacetate.

In some embodiments, the solvate form of Compound 1 provided herein is selected from an acetone solvate form, a methyl ethyl ketone solvate form, a dichloromethane solvate form, a tetrahydrofuran solvate form, a toluene solvate form, a n-butyl acetate solvate form, a methyl acetate solvate form, a xylene solvate form, a heptane solvate form, a 1-butanol solvate form, a p-dioxane solvate form, an ethyl acetate solvate form, a DMAc solvate form, and a dimethylacetamide solvate form.

In some embodiments, the solvate form is an acetone solvate form.

In some embodiments, the solvate form is acetone solvate, form I.

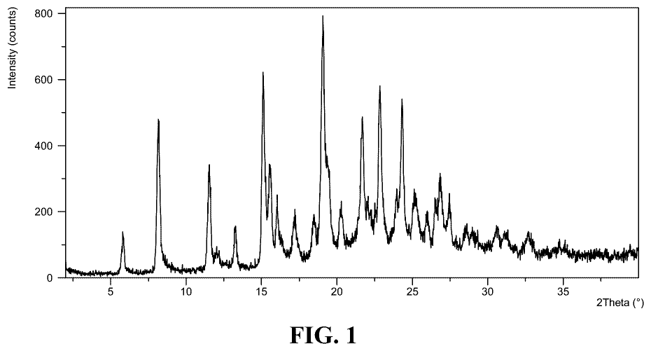

In some embodiments, the acetone solvate, form I is characterized by an XRPD pattern substantially as shown in FIG. 1.

In some embodiments, the solvate form is acetone solvate, form II.

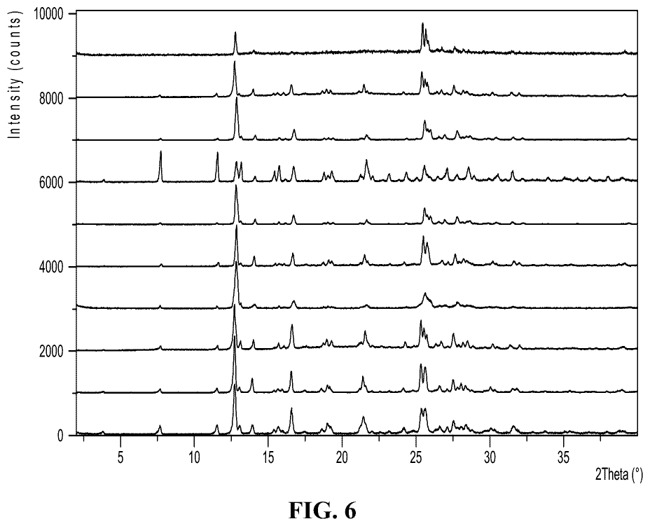

In some embodiments, the acetone solvate, form II is characterized by an XRPD pattern substantially as shown in FIG. 6.

In some embodiments, the solvate form is a methyl ethyl ketone solvate form.

In some embodiments, the methyl ethyl ketone solvate form is characterized by an XRPD pattern substantially as shown in FIG. 6.

In some embodiments, the solvate form is a dichloromethane solvate form.

In some embodiments, the dichloromethane solvate form is characterized by an XRPD pattern substantially as shown in FIG. 6.

In some embodiments, the solvate form is a tetrahydrofuran solvate form.

In some embodiments, the tetrahydrofuran solvate form is characterized by an XRPD pattern substantially as shown in FIG. 6.

In some embodiments, the solvate form is a toluene solvate form.

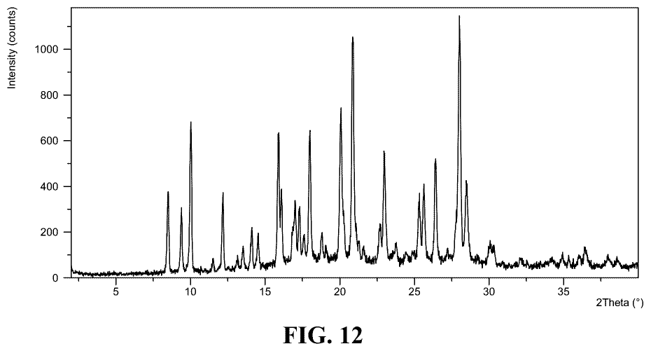

In some embodiments, the toluene solvate form is characterized by an XRPD pattern substantially as shown in FIG. 12.

In some embodiments, the solvate form is a n-butyl acetate solvate form.

In some embodiments, the n-butyl acetate solvate form is characterized by an XRPD pattern substantially as shown in FIG. 6.

In some embodiments, the solvate form is a methyl acetate solvate form.

In some embodiments, the methyl acetate solvate form is characterized by an XRPD pattern substantially as shown in FIG. 6.

In some embodiments, the solvate form is a xylene solvate form.

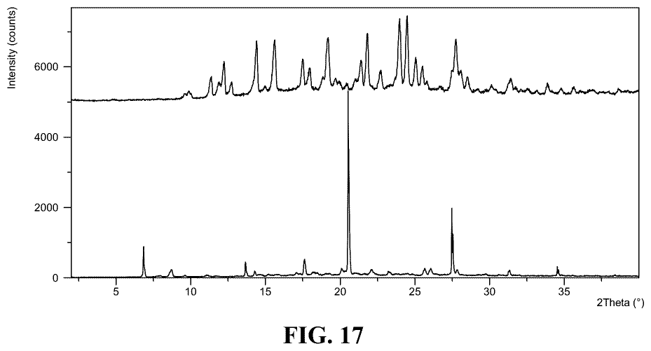

In some embodiments, the xylene solvate form is characterized by an XRPD pattern substantially as shown in FIG. 17.

In some embodiments, the solvate form is a heptane solvate form.

In some embodiments, the heptane solvate form is characterized by an XRPD pattern substantially as shown in FIG. 6.

In some embodiments, the solvate form is a 1-butanol solvate form.

In some embodiments, the 1-butanol solvate form is characterized by an XRPD pattern substantially as shown in FIG. 6.

In some embodiments, the solvate form is a p-dioxane solvate form.

In some embodiments, the p-dioxane solvate form is characterized by an XRPD pattern substantially as shown in FIG. 6.

In some embodiments, the solvate form is an ethyl acetate solvate form.

In some embodiments, the ethyl acetate solvate form is characterized by an XRPD pattern substantially as shown in FIG. 6.

In some embodiments, the solvate form is a dimethylacetamide solvate form.

In some embodiments, the dimethylacetamide solvate form is characterized by an XRPD pattern substantially as shown in FIG. 17.

According to the present invention, a crystalline form or solvate form of Compound 1 provided herein can be useful in the synthesis and/or purification of Compound 1. For example, a crystalline form of Compound 1 provided herein (e.g., crystalline Form II, crystalline Form III, crystalline Form IV, and crystalline Form V) can be an intermediate in the synthesis of Compound 1. In addition, different crystalline forms and solvate form of Compound 1 may have different properties with respect to bioavailability, stability, purity, and/or manufacturability for medical or pharmaceutical uses. Variations in the crystal structure of a pharmaceutical drug substance or active ingredient may affect the dissolution rate (which may affect bioavailability, etc.), manufacturability (e.g., ease of handling, ability to consistently prepare doses of known strength), and stability (e.g., thermal stability, shelf life, etc.) of a pharmaceutical drug product or active ingredient. Such variations may affect the preparation or formulation of pharmaceutical compositions in different dosage or delivery forms, such as solutions or solid oral dosage forms including tablets and capsules. Compared to other forms such as non-crystalline or amorphous forms, crystalline forms may provide desired or suitable hygroscopicity, particle size controls, dissolution rate, solubility, purity, physical and chemical stability, manufacturability, yield, and/or process control. Thus, the crystalline forms and/or solvate forms of Compound 1 provided herein may provide advantages such as improving the manufacturing process of the compound, the stability or storability of a drug product form of the compound, the stability or storability of a drug substance of the compound and/or the bioavailability and/or stability of the compound as an active agent.

The use of certain solvents and/or processes have been found to produce different crystalline forms and/or solvate forms of Compound 1, which may exhibit one or more of the favorable characteristics described above. The processes for the preparation of the crystalline forms and solvate forms described herein and characterization of these crystalline forms and solvate forms are described in detail below.

In some embodiments, the crystalline forms and solvate forms described herein, are purified or substantially isolated. By “substantially isolated,” it is meant that the crystalline form or solvate form is at least partially or substantially separated from the environment in which it was formed or detected. Partial separation can include, for example, a composition enriched in the crystalline form or solvate form of the invention. Substantial separation can include compositions containing at least about 50%, at least about 60%, at least about 70%, at least about 80%, at least about 90%, at least about 95%, at least about 97%, or at least about 99% by weight of the crystalline form or solvate form the invention. In some embodiments, the crystalline form or solvate form of the invention can be prepared with a purity of about 75% or more, 80% or more, 85% or more, 90% or more, 95% or more, 98% or more, or 99% or more.

Different crystalline forms and solvate forms can be identified by solid state characterization methods such as by X-ray powder diffraction (XRPD). Other characterization methods such as differential scanning calorimetry (DSC) further help identify the form as well as help determine stability and solvent/water content.

An XRPD pattern of reflections (peaks) is typically considered a fingerprint of a particular crystalline form. It is well known that the relative intensities of the XRPD peaks can widely vary depending on, inter alia, the sample preparation technique, crystal size distribution, various filters used, the sample mounting procedure, and the particular instrument employed. In some instances, new peaks may be observed or existing peaks may disappear, depending on the type of the instrument or the settings. As used herein, the term “peak” refers to a reflection having a relative height/intensity of at least about 5% of the maximum peak height/intensity. Moreover, instrument variation and other factors can affect the 2-theta values. Thus, peak assignments, such as those reported herein, can vary by plus or minus about 0.2° (2-theta), and the term “substantially” and “about” as used in the context of XRPD herein is meant to encompass the above-mentioned variations.

In the same way, temperature readings in connection with DSC can vary about +3° C. depending on the instrument, particular settings, sample preparation, etc. Accordingly, a crystalline form reported herein having a DSC thermogram “substantially” as shown in any of the Figures or the term “about” is understood to accommodate such variation.

The description below is made with the understanding that the present disclosure is to be considered as an exemplification of the claimed subject matter, and is not intended to limit the appended claims to the specific embodiments illustrated. The headings used throughout this disclosure are provided for convenience and are not to be construed to limit the claims in any way. Embodiments illustrated under any heading may be combined with embodiments illustrated under any other heading.

Unless defined otherwise, all technical and scientific terms used herein have the same meaning as commonly understood by one of ordinary skill in the art.

When trade names are used herein, it is intended to independently include the tradename product and the active pharmaceutical ingredient(s) of the tradename product.

As used herein and in the appended claims, the singular forms “a” and “an”, and “the” include plural referents unless the context clearly dictates otherwise. Thus, e.g., reference to “the compound” includes a plurality of such compounds and reference to “the assay” includes reference to one or more assays, and so forth.

“Pharmaceutically acceptable” refers to compounds, crystalline forms, solvate forms, compositions, dosage forms, and other materials which are useful in preparing a pharmaceutical composition that is suitable for veterinary or human pharmaceutical use.

“Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Subject” and “subjects” refers to humans, domestic animals (e.g., dogs and cats), farm animals (e.g., cattle, horses, sheep, goats and pigs), laboratory animals (e.g., mice, rats, hamsters, guinea pigs, pigs, rabbits, dogs, and monkeys), and the like.

As used herein, “treatment” or “treating” is an approach for obtaining beneficial or desired results. For purposes of the present disclosure, beneficial or desired results include, but are not limited to, alleviation of a symptom and/or diminishment of the extent of a symptom and/or preventing a worsening of a symptom associated with a disease or condition. In some embodiments, “treatment” or “treating” includes one or more of the following: a) inhibiting the disease or condition (e.g., decreasing one or more symptoms resulting from the disease or condition, and/or diminishing the extent of the disease or condition); b) slowing or arresting the development of one or more symptoms associated with the disease or condition (e.g., stabilizing the disease or condition, delaying the worsening or progression of the disease or condition); and/or c) relieving the disease or condition, e.g., causing the regression of clinical symptoms, ameliorating the disease state, delaying the progression of the disease, increasing the quality of life, and/or prolonging survival.

As used herein, “delaying” development of a disease or condition means to defer, hinder, slow, retard, stabilize and/or postpone development of the disease or condition. This delay can be of varying lengths of time, depending on the history of the disease and/or subject being treated. As is evident to one skilled in the art, a sufficient or significant delay can, in effect, encompass prevention, in that the subject does not develop the disease or condition. For example, a method that “delays” development of AIDS is a method that reduces the probability of disease development in a given time frame and/or reduces extent of the disease in a given time frame, when compared to not using the method. Such comparisons may be based on clinical studies, using a statistically significant number of subjects. For example, the development of AIDS can be detected using known methods, such as confirming a subject's HIV+ status and assessing the subject's T-cell count or other indication of AIDS development, such as extreme fatigue, weight loss, persistent diarrhea, high fever, swollen lymph nodes in the neck, armpits or groin, or presence of an opportunistic condition that is known to be associated with AIDS (e.g., a condition that is generally not present in subjects with functioning immune systems but does occur in AIDS patients). Development may also refer to disease progression that may be initially undetectable and includes occurrence, recurrence and onset.

As used herein, “prevention” or “preventing” refers to a regimen that protects against the onset of the disease or disorder such that the clinical symptoms of the disease do not develop. Thus, “prevention” relates to administration of a therapy (e.g., administration of a therapeutic substance) to a subject before signs of the disease are detectable in the subject (e.g., administration of a therapeutic substance to a subject in the absence of detectable infectious agent (e.g., virus) in the subject). The subject may be an individual at risk of developing the disease or disorder, such as an individual who has one or more risk factors known to be associated with development or onset of the disease or disorder. Thus, the term “preventing HIV infection” refers to administering to a subject who does not have a detectable HIV infection an anti-HIV therapeutic substance. It is understood that the subject for anti-HIV preventative therapy may be an individual at risk of contracting the HIV virus. Further, it is understood that prevention may not result in complete protection against onset of the disease or disorder. In some instances, prevention includes reducing the risk of developing the disease or disorder. The reduction of the risk may not result in complete elimination of the risk of developing the disease or disorder.

As used herein, an “at risk” individual is an individual who is at risk of developing a condition to be treated. An individual “at risk” may or may not have detectable disease or condition, and may or may not have displayed detectable disease prior to the treatment of methods described herein. “At risk” denotes that an individual has one or more so-called risk factors, which are measurable parameters that correlate with development of a disease or condition and are known in the art. An individual having one or more of these risk factors has a higher probability of developing the disease or condition than an individual without these risk factor(s). For example, individuals at risk for AIDS are those having HIV.

As used herein, the term “therapeutically effective amount” or “effective amount” refers to an amount that is effective to elicit the desired biological or medical response, including the amount of a compound that, when administered to a subject for treating a disease, is sufficient to effect such treatment for the disease or to an amount that is effective to protect against the contracting or onset of a disease. The effective amount will vary depending on the compound, the disease, and its severity and the age, weight, etc., of the subject to be treated. The effective amount can include a range of amounts. As is understood in the art, an effective amount may be in one or more doses, i.e., a single dose or multiple doses may be required to achieve the desired treatment outcome. A single agent may be considered to be given in an effective amount if a desirable or beneficial result may be or is achieved.

“Enantiomers” are a pair of stereoisomers that are non-superimposable mirror images of each other. A 1:1 mixture of a pair of enantiomers is a “racemic” mixture. A mixture of enantiomers at a ratio other than 1:1 is a “scalemic” mixture.

“Diastereoisomers” are stereoisomers that have at least two asymmetric atoms, but which are not mirror-images of each other.

The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds and salts described herein contain one or more asymmetric centers and/or hindered rotation about a bond axis and may thus give rise to enantiomers, diastereomers, and other stereoisomeric forms that may be defined, in terms of absolute stereochemistry, as (R)- or (S)-. The present disclosure is meant to include all such possible isomers, including racemic mixtures, scalemic mixtures, diastereomeric mixtures, optically pure forms and intermediate mixtures. Optically active (R)- and (S)-isomers may be prepared using chiral synthons or chiral reagents, or resolved using conventional techniques.

Except as expressly defined otherwise, the present disclosure includes all tautomers of compounds detailed herein, even if only one tautomer is expressly represented (e.g., both tautomeric forms are intended and described by the presentation of one tautomeric form where a pair of two tautomers may exist). For example, if reference is made to a compound containing an amide (e.g., by structure or chemical name), it is understood that the corresponding imidic acid tautomer is included by this disclosure and described the same as if the amide were expressly recited either alone or together with the imidic acid. Where more than two tautomers may exist, the present disclosure includes all such tautomers even if only a single tautomeric form is depicted by chemical name and/or structure.

It is understood by one skilled in the art that this disclosure also includes any salt disclosed herein and may be enriched at any or all atoms above naturally occurring isotopic ratios with one or more isotopes such as, but not limited to, deuterium (²H or D).

Disclosed are crystalline forms and solvate forms of Compound 1 in which from 1 to n hydrogen atoms attached to a carbon atom may be replaced by a deuterium atom or D, in which n is the number of hydrogen atoms in the molecule. As known in the art, the deuterium atom is a non-radioactive isotope of the hydrogen atom. Such salts may increase resistance to metabolism, and thus may be useful for increasing the half-life of the compounds when administered to a mammal. See, e.g., Foster. “Deuterium Isotope Effects in Studies of Drug Metabolism”, Trends Pharmacol. Sci., 5(12):524-527 (1984). Such salts are synthesized by means well known in the art, for example by employing starting materials in which one or more hydrogen atoms have been replaced by deuterium.

Examples of isotopes that can be incorporated into the disclosed salts also include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled salts can generally be prepared by conventional techniques known to those skilled in the art or by processes analogous to those described in the Examples as set out below using an appropriate isotopically-labeled reagent in place of the non-labeled reagent previously employed.

Compounds described herein may have chiral centers and/or geometric isomeric centers (E- and Z-isomers), and it is to be understood that all such optical, enantiomeric, diastereoisomeric and geometric isomers are encompassed. Where compounds are represented in their chiral form, it is understood that the embodiment encompasses, but is not limited to, the specific diastereomerically or enantiomerically enriched form. Where chirality is not specified but is present, it is understood that the embodiment is directed to either the specific diastereomerically or enantiomerically enriched form; or a racemic or scalemic mixture of such compound(s).

In some embodiments, the current disclosure relates to the use of the crystalline forms or solvate forms of the invention in treating a Retroviridae viral infection including an infection caused by the HIV virus comprising administering a therapeutically effective amount of the crystalline form or solvate form to a subject in need thereof.

It is a desirable goal to discover a compound, or crystalline form or solvate form thereof having a low EC₅₀. The EC₅₀ value refers to the concentration of a compound in an assay that achieves 50% of the maximum efficacy. A compound, crystalline form, or solvate form with a lower EC₅₀ achieves similar efficacy with lower compound, crystalline form, or solvate form concentration relative to a compound, crystalline form, or solvate form with a higher EC₅₀. Thus, a lower EC₅₀ is generally preferred for drug development.

It is a desirable goal to discover a compound, crystalline form, or solvate form that has good physical and/or chemical stability. An increase in overall stability of a compound, crystalline form, or solvate form can provide an increase in circulation time in the body. With less degradation, a stable compound, crystalline form, or solvate form can be administered in lower doses and still maintain efficacy. Also, with less degradation, there is less concern about by-products from degradation of a compound, crystalline form, or solvate form.

It is a desirable goal to discover a compound, crystalline form, or solvate form thereof that has improved pharmacokinetic and/or pharmacodynamic profiles and long half-life. It is advantageous for a drug to have a moderate or low clearance and a long half-life, as this can lead to a good bioavailability and high exposure in systemic exposure. Reducing the clearance and increasing half-life time of a compound, crystalline form, or solvate form could reduce the daily dose required for efficacy and therefore give a better efficacy and safety profile. Thus, improved pharmacokinetic and/or pharmacodynamic profiles and long half-life can provide for better patient compliance.

### Methods of Use

In some embodiments, the crystalline forms or solvate forms disclosed herein are used for preventing an HIV infection in a subject. In some embodiments, the crystalline forms or solvate forms disclosed herein are used for preventing an HIV infection in a subject at risk for infection. In some embodiments, the crystalline forms or solvate forms disclosed herein are used for pre-exposure prophylaxis (PrEP) to reduce the risk of sexually acquired HIV-1.

In certain embodiments, a method for treating or preventing an HIV infection in a subject (e.g., a human), comprising administering a crystalline form or solvate form of Compound 1, to the subject is disclosed.

In certain embodiments, a method for inhibiting the replication of the HIV virus, treating AIDS or delaying the onset of AIDS in a subject (e.g., a human), comprising administering a crystalline form or solvate form of Compound 1, to the subject is disclosed.

In some embodiments, a method for inhibiting the replication of the HIV virus, treating AIDS or delaying the onset of AIDS in a subject (e.g., a human), comprising administering a crystalline form or solvate form of Compound 1, to the subject is disclosed.

In certain embodiments, a method for preventing an HIV infection in a subject (e.g., a human), comprising administering a crystalline form or solvate form of Compound 1, to the subject is disclosed. In certain embodiments, the subject is at risk of contracting the HIV virus, such as a subject who has one or more risk factors known to be associated with contracting the HIV virus.

In some embodiments, a method for preventing an HIV infection in a subject (e.g., a human), comprising administering a therapeutically effective amount of a crystalline form or solvate form of Compound 1, to the subject is disclosed. In certain embodiments, the subject is at risk of contracting the HIV virus, such as a subject who has one or more risk factors known to be associated with contracting the HIV virus.

In some embodiments, a method for treating an HIV infection in a subject (e.g., a human), comprising administering a crystalline form of Compound 1, to the subject is disclosed.

In some embodiments, a method for treating an HIV infection in a subject (e.g., a human), comprising administering a solvate form of Compound 1, to the subject is disclosed.

In some embodiments, a crystalline form of Compound 1, for use in medical therapy of an HIV infection (e.g., HIV-1 or the replication of the HIV virus (e.g., HIV-1) or AIDS or delaying the onset of AIDS in a subject (e.g., a human)) is disclosed.

In some embodiments, a solvate form of Compound 1, for use in medical therapy of an HIV infection (e.g., HIV-1 or the replication of the HIV virus (e.g. HIV-1) or AIDS or delaying the onset of AIDS in a subject (e.g., a human)) is disclosed.

In some embodiments, a crystalline form or solvate form of Compound 1, for use in the manufacture of a medicament for treating an HIV infection or the replication of the HIV virus or AIDS or delaying the onset of AIDS in a subject (e.g., a human) is disclosed. In some embodiments, a crystalline form or solvate form of Compound 1, for use in the prophylactic or therapeutic treatment of an HIV infection or AIDS or for use in the therapeutic treatment or delaying the onset of AIDS is disclosed.

In some embodiments, a crystalline form of Compound 1 for use in the manufacture of a medicament for treating an HIV infection or the replication of the HIV virus or AIDS or delaying the onset of AIDS in a subject (e.g., a human) is disclosed. In some embodiments, a crystalline form of Compound 1 for use in the prophylactic or therapeutic treatment of an HIV infection or AIDS or for use in the therapeutic treatment or delaying the onset of AIDS is disclosed.

In some embodiments, a solvate form of Compound 1 for use in the manufacture of a medicament for treating an HIV infection or the replication of the HIV virus or AIDS or delaying the onset of AIDS in a subject (e.g., a human) is disclosed. In some embodiments, a solvate form of Compound 1 for use in the prophylactic or therapeutic treatment of an HIV infection or AIDS or for use in the therapeutic treatment or delaying the onset of AIDS is disclosed.

In certain embodiments, a crystalline form or solvate form of Compound 1, for the manufacture of a medicament for an HIV infection in a subject (e.g., a human) is disclosed. In certain embodiments, a crystalline form or solvate form of Compound 1, for use in the prophylactic or therapeutic treatment of an HIV infection is disclosed.

In certain embodiments, a crystalline form Compound 1 for the manufacture of a medicament for an HIV infection in a subject (e.g., a human) is disclosed. In certain embodiments, a crystalline form of Compound 1 for use in the prophylactic or therapeutic treatment of an HIV infection is disclosed.

In certain embodiments, a solvate form of Compound 1, for the manufacture of a medicament for an HIV infection in a subject (e.g., a human) is disclosed. In certain embodiments, a solvate form of Compound 1, for use in the prophylactic or therapeutic treatment of an HIV infection is disclosed.

In certain embodiments, in the methods of use, the administration is to a subject (e.g., a human) in need of the treatment. In certain embodiments, in the methods of use, the administration is to a subject (e.g., a human) who is at risk of developing AIDS.

Disclosed herein is a crystalline form or solvate form of Compound 1, for use in therapy. In some embodiments, the crystalline form of Compound 1, is for use in a method of treating an HIV infection or the replication of the HIV virus or AIDS or delaying the onset of AIDS in a subject (e.g., a human). In some embodiments, the solvate form of Compound 1, is for use in a method of treating an HIV infection or the replication of the HIV virus or AIDS or delaying the onset of AIDS in a subject (e.g., a human).

Also disclosed herein is a crystalline form or solvate form of Compound 1, for use in a method of treating or preventing HIV infection in a subject in need thereof. In certain embodiments, a crystalline form or solvate form of Compound 1, for use in a method of treating HIV infection in a subject in need thereof is provided. In certain embodiments, the subject in need thereof is a human who has been infected with HIV. In certain embodiments, the subject in need thereof is a human who has been infected with HIV but who has not developed AIDS. In certain embodiments, the subject in need thereof is a subject at risk for developing AIDS. In certain embodiments, the subject in need thereof is a human who has been infected with HIV and who has developed AIDS.

In some embodiments, disclosed herein is a crystalline form or solvate form of Compound 1, for use in a method of treating or preventing HIV infection in a subject in need thereof. In certain embodiments, a crystalline form or solvate form of Compound 1, for use in a method of treating HIV infection in a subject in need thereof is provided. In certain embodiments, the subject in need thereof is a human who has been infected with HIV. In certain embodiments, the subject in need thereof is a human who has been infected with HIV but who has not developed AIDS. In certain embodiments, the subject in need thereof is a subject at risk for developing AIDS. In certain embodiments, the subject in need thereof is a human who has been infected with HIV and who has developed AIDS.

In some embodiments, a crystalline form or solvate form of Compound 1, is provided for use to prevent HIV infection from taking hold if the individual is exposed to the virus and/or to keep the virus from establishing a permanent infection and/or to prevent the appearance of symptoms of the disease and/or to prevent the virus from reaching detectable levels in the blood, for example for pre-exposure prophylaxis (PrEP) or post-exposure prophylaxis (PEP). Accordingly, in certain embodiments, methods for reducing the risk of acquiring HIV (e.g., HIV-1 and/or HIV-2) are provided. For example, methods for reducing the risk of acquiring HIV (e.g., HIV-1 and/or HIV-2) comprise administration of a crystalline form or solvate form of Compound 1. In certain embodiments, methods for reducing the risk of acquiring HIV (e.g., HIV-1 and/or HIV-2) comprise administration of a pharmaceutical composition comprising a therapeutically effective amount of a crystalline form or solvate form of Compound 1, and one or more excipients.

In certain embodiments, methods for reducing the risk of acquiring HIV (e.g., HIV-1 and/or HIV-2) comprise administration of a crystalline form or solvate form of Compound 1, in combination with safer sex practices. In certain embodiments, methods for reducing the risk of acquiring HIV (e.g., HIV-1 and/or HIV-2) comprise administration to an individual at risk of acquiring HIV. Examples of individuals at high risk for acquiring HIV include, without limitation, an individual who is at risk of sexual transmission of HIV.

In certain embodiments, the reduction in risk of acquiring HIV is at least about 40%, 50%, 60%, 70%, 80%, 90%, or 95%. In certain embodiments, the reduction in risk of acquiring HIV is at least about 75%. In certain embodiments, the reduction in risk of acquiring HIV is about 80%, 85%, or 90%.

In some embodiments, the use of a crystalline form or solvate form of Compound 1, for the manufacture of a medicament for the treatment of an HIV infection in a human being having or at risk of having the infection is disclosed.

In some embodiments, disclosed herein is a crystalline form or solvate form of Compound 1, for use in the therapeutic treatment or delaying the onset of AIDS.

In certain embodiments, a crystalline form or solvate form of Compound 1 can be used as a research tool.

### Routes of Administration

The crystalline form or solvate form of Compound 1, (also referred to herein as the active ingredient) can be administered by any route appropriate to the condition to be treated. Suitable routes include oral, rectal, nasal, topical (including buccal and sublingual), transdermal, vaginal and parenteral (including subcutaneous, intramuscular, intravenous, intradermal, intrathecal and epidural), and the like. It will be appreciated that the preferred route may vary with, for example, the condition of the recipient. In certain embodiments, the compound can be dosed parenterally. In certain embodiments, the compound disclosed can be dosed intravenous, subcutaneous, or intramuscular. In certain embodiments, the crystalline form or solvate form of Compound 1 are orally bioavailable and can be dosed orally.

In some embodiments, the crystalline form or solvate form of Compound 1, may be administered via injection, using an injection device. In some embodiments, the injection device is or includes a syringe, which can be employed manually, or as part of a syringe-containing injection device, such as, but not limited to, one with a needle safety shield. A wide variety of injection devices can be used, such as, for example and not limited to, a handheld or wearable autoinjector, a handheld or wearable manual injector, an on-body injector, a syrette, a jet injector, or a pen injector, each of which can be reusable or disposable.

In some embodiments, the crystalline form or solvate form of Compound 1 may be administered with an auto-injector comprising a syringe. In some embodiments, the syringe is disposable. In some embodiments, the syringe is reusable. In some embodiments, the syringe is pre-filled with the crystalline form or solvate form of Compound 1.

### Dosing Regimen

The crystalline form or solvate form of Compound 1 may be administered to a subject (e.g., a human) in an effective amount. In some embodiments, the crystalline form or solvate form of Compound 1 may be administered to a subject (e.g., a human) in an therapeutically effective amount.

The crystalline form or solvate form of Compound 1 may be administered to a subject in accordance with an effective dosing regimen for a desired period of time or duration, such as at least about one day, at least about one week, at least about one month, at least about 2 months, at least about 3 months, at least about 4 months, at least about 6 months, or at least about 12 months or longer. In some embodiments, the crystalline form or solvate form of Compound 1 is administered on a daily or intermittent schedule. In certain embodiments, the crystalline form or solvate form of Compound 1 is administered once daily. In some embodiments, the crystalline form or solvate form of Compound 1 is administered on a monthly schedule. In some embodiments, the crystalline form or solvate form of Compound 1 is administered every two months. In some embodiments, the crystalline form or solvate form of Compound 1 is administered every three months. In some embodiments, the crystalline form or solvate form of Compound 1 is administered every four months. In some embodiments, the crystalline form or solvate form of Compound 1 is administered every five months. In some embodiments, the crystalline form or solvate form of Compound 1 is administered every six months.

The dosage or dosing frequency of the crystalline form or solvate form of Compound 1 may be adjusted over the course of the treatment, based on the judgment of the administering physician.

A crystalline form or solvate form of Compound 1 disclosed herein may be administered in a dosage amount that is effective. For example, the dosage amount can be from 1 mg to 1000 mg of compound. In certain embodiments, the dosage amount is about 1, 10, 20, 30, 40, 50, 60, 70, 80, 90, 95, 100, 105, 110, 120, 130, 140, or 150 mg of compound. In certain embodiments the dosage amount is about 100, 150, 200, 250, 300, 350, 400, 450, 500, 550, 600, 650, 700, 750, 800, 850, 900, 950, or 1000 mg.

In some embodiments, the crystalline form or solvate form of Compound 1 is administered in a once daily dose.

### Pharmaceutical Compositions

Pharmaceutical compositions disclosed herein comprise a crystalline form or solvate form of Compound 1, disclosed herein together with one or more pharmaceutically acceptable excipients. Pharmaceutical compositions containing the active ingredient may be in any form suitable for the intended method of administration.

In some embodiments, pharmaceutical compositions disclosed herein comprise a crystalline form or solvate form of Compound 1 disclosed herein together with one or more pharmaceutically acceptable excipients. Pharmaceutical compositions containing the active ingredient may be in any form suitable for the intended method of administration.

Pharmaceutical compositions comprising the crystalline form or solvate form of Compound 1, disclosed herein may be prepared with conventional carriers (e.g., inactive ingredient or excipient material) which may be selected in accord with ordinary practice. Tablets may contain excipients including glidants, fillers, binders and the like. Aqueous compositions may be prepared in sterile form, and when intended for delivery by other than oral administration generally may be isotonic. All compositions may optionally contain excipients such as those set forth in the Rowe et al, Handbook of Pharmaceutical Excipients, 5th edition, American Pharmacists Association, 1986. Excipients can include ascorbic acid and other antioxidants, chelating agents such as EDTA, carbohydrates such as dextrin, hydroxyalkylcellulose, hydroxyalkylmethylcellulose, stearic acid and the like.

While it is possible for the active ingredient to be administered alone, it may be preferable to present the active ingredient as pharmaceutical compositions. The compositions, both for veterinary and for human use, comprise at least the crystalline form or solvate form of Compound 1, disclosed herein together with one or more acceptable carriers. The carrier(s) are “acceptable” in the sense of being compatible with the other ingredients of the composition and physiologically innocuous to the recipient thereof.

The compositions include those suitable for various administration routes. The compositions may conveniently be presented in unit dosage form and may be prepared by any of the methods well known in the art of pharmacy. Such methods include the step of bringing into association the active ingredient with one or more inactive ingredients (e.g., a carrier, pharmaceutical excipient, etc.). The compositions may be prepared by uniformly and intimately bringing into association the active ingredient with liquid carriers or finely divided solid carriers or both, and then, if necessary, shaping the product. Techniques and formulations generally are found in Remington: The Science and Practice of Pharmacy, 21^{st} Edition, Lippincott Williams and Wilkins, Philadelphia, Pa., 2006.

Compositions described herein that are suitable for oral administration may be presented as discrete units (a unit dosage form) including but not limited to capsules, cachets or tablets each containing a predetermined amount of the active ingredient.

When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, lactose monohydrate, croscarmellose sodium, povidone, calcium or sodium phosphate; granulating and disintegrating agents, such as maize starch, or alginic acid; binding agents, such as cellulose, microcrystalline cellulose, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

In some embodiments, disclosed herein are oral dosage forms (e.g., tablets), which may be prepared from hot melt extrusion or spray-drying dispersion (SDD) technologies.

In some embodiments, disclosed herein are hard capsules filled with powder, beads, or granules containing the active ingredient in admixture with non-toxic pharmaceutically acceptable excipient which are suitable for manufacture of hard or soft capsules. These excipients may be, for example, inert diluents, such as calcium or sodium carbonate, lactose, lactose monohydrate, croscarmellose sodium, povidone, calcium or sodium phosphate; granulating and disintegrating agents, such as maize starch, or alginic acid, binding agents, such as cellulose, microcrystalline cellulose, starch, gelatin or acacia; and lubricating agents, such as magnesium stearate, stearic acid or talc.

In some embodiments, disclosed herein are hard or soft capsules filled with liquid or semi-solid mixtures containing the active ingredient in admixture with non-toxic pharmaceutically acceptable excipient which are suitable for manufacture of hard or soft capsules. These excipients may be, for example, solubilizing oils such as maize oil, sesame oil, or corn oil; medium chain triglycerides and related esters, such as, derivitized palm kernel oil or coconut oil; self-emulsifying lipid systems (SEDDS or SMEDDS), such as caprylic triglyceride or propylene glycol monocaprylate; viscosity modifiers, such as, cetyl alcohol, steryl alcohol, glycerol stearate; and solubilizing agents and surfactants, such as polyethylene glycol, propylene glycol, glycerin, ethanol, polyethoxylated castor oil, poloxamers, or polysorbates.

The pharmaceutical compositions of the present disclosure may be in the form of a sterile injectable preparation, such as a sterile injectable aqueous or oleaginous suspension. This suspension may be formulated according to the known art using those suitable dispersing or wetting agents and suspending agents which have been mentioned herein. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

In some embodiments, the sterile injectable preparation disclosed herein may also be a sterile injectable solution or suspension prepared from a reconstituted lyophilized powder in a non-toxic parenterally acceptable diluent or solvent, such as a solution in 1,3-butane-diol. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. In certain embodiments the suspension is a microsuspension. In certain embodiments the suspension is a nanosuspension.

In some embodiments, formulations suitable for parenteral administration (e.g., intramuscular (IM) and subcutaneous (SC) administration) will include one or more excipients. Excipients should be compatible with the other ingredients of the formulation and physiologically innocuous to the recipient thereof. Examples of suitable excipients are well known to the person skilled in the art of parenteral formulation and may be found e.g., in Handbook of Pharmaceutical Excipients (eds. Rowe, Sheskey & Quinn), 6th edition 2009.

Examples of solubilizing excipients in a parenteral formulation include, but are not limited to, polysorbates (such as polysorbate 20 or 80) and poloxamers (such as poloxamer 338, 188, or 207).

In some embodiments, the parenteral formulation disclosed herein is an aqueous suspension. In some embodiments, the parenteral formulation disclosed herein is an aqueous suspension that comprises a crystalline form or solvate form of Compound 1 disclosed herein and saline. In some embodiments, the parenteral formulation disclosed herein is an aqueous suspension that comprises a crystalline form or solvate form of Compound 1 disclosed herein, saline, and a poloxamer.

In certain embodiments, the composition is disclosed as a solid dosage form, including a solid injectable dosage form, such as a solid depot form.

The amount of active ingredient that may be combined with the inactive ingredients to produce a dosage form may vary depending upon the intended treatment subject and the particular mode of administration. For example, in some embodiments, a dosage form for oral administration to humans may contain approximately 1 to 1000 mg of active material formulated with an appropriate and convenient amount of carrier material (e.g., inactive ingredient or excipient material). In certain embodiments, the carrier material varies from about 5 to about 95% of the total compositions (weight:weight).

It should be understood that in addition to the ingredients particularly mentioned above the compositions of these embodiments may include other agents conventional in the art having regard to the type of composition in question, for example those suitable for oral administration may include flavoring agents.

In certain embodiments, a composition comprising an active ingredient disclosed herein in one variation does not contain an agent that affects the rate at which the active ingredient is metabolized. Thus, it is understood that compositions comprising a crystalline form or solvate form of Compound 1, in certain embodiments do not comprise an agent that would affect (e.g., slow, hinder or retard) the metabolism of the crystalline form or solvate form of Compound 1. It is also understood that any of the methods, kits, articles of manufacture and the like detailed herein in certain embodiments do not comprise an agent that would affect (e.g., slow, hinder or retard) the metabolism of a crystalline form or solvate form of Compound 1.

### Kits and Articles of Manufacture

The present disclosure relates to a kit comprising a crystalline form or solvate form of Compound 1 disclosed herein. The kit may further comprise instructions for use, e.g., for use in inhibiting an HIV reverse transcriptase, such as for use in treating an HIV infection or AIDS or as a research tool. The instructions for use are generally written instructions, although electronic storage media (e.g., magnetic diskette or optical disk) containing instructions are also acceptable.

The present disclosure also relates to a pharmaceutical kit comprising one or more containers comprising a crystalline form or solvate form of Compound 1 disclosed herein. Optionally associated with such container(s) can be a notice in the form prescribed by a governmental agency regulating the manufacture, use or sale of pharmaceuticals, which notice reflects approval by the agency for the manufacture, use or sale for human administration. Each component (if there is more than one component) can be packaged in separate containers or some components can be combined in one container where cross-reactivity and shelf life permit. The kits may be in unit dosage forms, bulk packages (e.g., multi-dose packages) or sub-unit doses. Kits may also include multiple unit doses of the compound and instructions for use and be packaged in quantities sufficient for storage and use in pharmacies (e.g., hospital pharmacies and compounding pharmacies).

In some embodiments, the present disclosure also relates to a pharmaceutical kit comprising one or more containers comprising a crystalline form or solvate form of Compound 1 disclosed herein. Optionally associated with such container(s) can be a notice in the form prescribed by a governmental agency regulating the manufacture, use or sale of pharmaceuticals, which notice reflects approval by the agency for the manufacture, use or sale for human administration.

Also disclosed are articles of manufacture comprising a unit dosage of a crystalline form or solvate form of Compound 1 disclosed herein in suitable packaging for use in the methods described herein. Suitable packaging is known in the art and includes, for example, vials, vessels, ampules, bottles, jars, flexible packaging and the like. An article of manufacture may further be sterilized and/or sealed.

The following abbreviations may be used herein:
° C. Degree Celsius
DSC Differential scanning calorimetry
DMAc Dimethylacetamide
DVS Dynamic Vapor Sorption
SCXRD Single Crystal X-Ray Diffraction
TGA Thermogravimetric analysis
XRPD X-Ray powder diffraction

### EXAMPLES

### General Materials and Methods

The following materials and methods were used throughout the Examples:

X-ray powder diffraction (XRPD) analysis was conducted on a diffractometer (PANalytical XPERT-PRO, PANalytical B. V., Almelo, Netherlands) using copper radiation (Cu Kα, λ=1.541874). Samples were spread evenly on a zero-background sample plate. The generator was operated at a voltage of 45 kV and amperage of 40 mA. Slits were Soller 0.02 rad, antiscatter 1.0°, and divergence. Scans were performed from 2 to 40° 2θ with a 0.0167 step size. Data analysis was performed using X'Pert Viewer V1.9a (PANalytical B.V., Almelo. Netherlands).

Differential Scanning Calorimetry (DSC) and Modulated Differential Scanning Calorimetry (MDSC) were run on Model Q2000 (TA Instruments, New Castle, DE). About 1-5 mg of material was loaded into a Tzero standard aluminum pan with a manually punctured pinhole on the lid. By default, the sample and reference pans were heated from 20 to 300° C. under nitrogen purge of 50 mL/min. DSC was run at a heating rate of 10° C./min, while MDSC was run at a heating rate of 5° C./min with modulation of ±1.00° C. every 60 s. Data analysis was completed using Universal Analysis 2000 Version 4.5A (TA Instruments, New Castle, DE).

Thermogravimetric Analysis (TGA) was used to evaluate sample weight loss as a function of temperature on either Model Q5000 or Q500 (TA Instruments, New Castle, DE). About 1 to 5 mg of material was loaded onto a sample pan and the sample was heated from ambient temperature to 300° C. or above at a rate of 10° C./min. The sample pan was under nitrogen purge at 40 mL/min. Data analysis was completed using Universal Analysis 2000 Version 4.5A (TA Instruments, New Castle, DE).

Dynamic Vapor Sorption (DVS) was used to study hygroscopicity on Model Q5000 SA (TA Instruments, New Castle, DE). A sample (1-10 mg) was placed in an aluminum pan and loaded on the sample side of the twin pan balance. The water sorption and desorption were studied as a function of relative humidity (RH) at 25° C. in 10% RH increments from 0% RH to 90% RH and then back to 0%. Each relative humidity increment had an equilibration time of 120 minutes unless weight change was less than 0.002% in 20 minutes. Data analysis was performed using Universal Analysis 2000 Version 4.7A (TA Instruments, New Castle, DE).

### Example 1. Preparation of Compound 1, Crystalline Form II

To a 4-mL vial equipped with a Teflon coated magnetic stir bar, about 100 mg of Compound 1, free base crystalline Form I (see e.g., U.S. Publication Nos.: 20220323476A1 and 20220332751A1, the disclosures of each of which are incorporated herein by reference in their entireties) was stirred with about 0.5 mL acetone at about 22° C. After three days. XRPD analysis of the wet solids showed a new pattern, designated as Compound 1, acetone solvate 1 (see FIG. 1). After drying at 50° C. under vacuum, the XRPD pattern changed and the resulting new form was designated as Compound 1, crystalline Form II.

A representative XRPD pattern for Compound 1, crystalline Form II is shown in FIG. 2 and is characterized by sharp reflections, indicating crystallinity. An XRPD peaking listing of Compound 1, crystalline Form II is provided in Table 1.

TABLE 1

| 2-Theta (°) | Relative Intensity (%) |
|---|---|
| 5.5 | 8 |
| 9.3 | 21 |
| 10.8 | 48 |
| 11.9 | 9 |
| 12.6 | 13 |
| 13.9 | 4 |
| 14.9 | 100 |
| 15.2 | 42 |
| 15.7 | 44 |
| 16.2 | 5 |
| 17.4 | 21 |
| 18.1 | 13 |
| 18.5 | 80 |
| 19.3 | 71 |
| 19.8 | 12 |
| 20.4 | 6 |
| 21.6 | 21 |
| 21.9 | 27 |
| 23.1 | 6 |
| 23.8 | 56 |
| 24.3 | 45 |
| 24.8 | 27 |
| 25.4 | 42 |
| 26.0 | 33 |
| 27.2 | 6 |
| 27.9 | 12 |
| 28.4 | 47 |
| 29.9 | 7 |
| 30.3 | 7 |
| 33.2 | 5 |
| 34.5 | 5 |

The DSC thermogram indicated a small endothermic transition at about 93° C. attributed to a form change from Compound 1, crystalline Form II to Compound 1, crystalline Form V (confirmed by XRPD in another experiment), which has a melting onset at about 156° C., as shown in FIG. 3. The TGA thermogram showed no substantial weight loss (0.01% from 25 to 150° C.), indicating no residual solvent, as shown in FIG. 4. The DVS analysis is shown in FIG. and indicated that the form is non-hygroscopic showing around 0.1% weight change from 0 to 90% RH at 25° C.

### Example 2. Preparation of Compound 1, Crystalline Form III

To a 4-mL vial equipped with a Teflon coated magnetic stir bar, about 100 mg of Compound 1, free base crystalline Form I was stirred with about 0.5 mL dichloromethane (DCM) at about 22° C. After three days, XRPD analysis of the wet solids that was covered with a Kapton film showed a new pattern, designated as Compound 1, DCM solvate (see FIG. 6). After drying in air, the XRPD pattern changed and the resulting new form was designated as Compound 1, crystalline Form III.

Alternatively. Compound 1, crystalline Form III can be obtained by drying other Compound 1 solvate forms, such as acetone solvate 2, methyl ethyl ketone, ethyl acetate, methyl acetate, n-butyl acetate, tetrahydrofuran, 1-butanol, or p-dioxane solvate at 50° C. under vacuum. Representative XRPD patterns of these solvate forms are shown in FIG. 6.

Single crystals of Compound 1, dichloromethane (DCM) solvate were prepared by dissolving about 105 mg of Compound 1 in 0.5 mL of DCM solution at about 40° C. and then held at about 22° C. for weeks. A slurry formed and the sample was subjected to SCXRD analysis at about 298° C. Because DCM was volatile, it escaped from the crystal lattice and the result was consistent with Compound 1, Form III with the following crystal parameters.

TABLE 2

| | |
|---|---|
| Temperature | 298 (5) K |
| Crystal system | Monoclinic |
| Space group | P2₁ |
| Unit cell | a = 6.95150(10) Å |
| dimensions | b = 8.30050(10) Å |
| | c = 23.2335(4) Å |
| | α = 90° |
| | β = 97.019(2)° |
| | γ = 90° |
| Z | 2 |
| Calculated density | 1.322 |
| (g/cm⁻³) | |
| Goodness-of-fit on | 1.06 |
| F2 | |

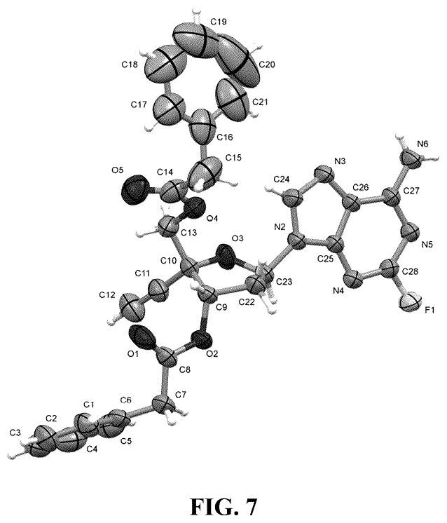

The molecule observed in the asymmetric unit of the single crystal structure was consistent with the molecular structure of Compound 1. The asymmetric unit shown in FIG. 7 contains one Compound 1 molecule. One of the phenylacetate moieties was disordered (as shown by dots), refining to 73% occupancy in the predominant orientation. Void space sufficient for solvent was present at the vacated orientations of the phenylacetate.

A representative XRPD pattern for Compound 1, crystalline Form III obtained after drying the DCM solvate is shown in FIG. 8 and is characterized by sharp reflections, indicating crystallinity. An XRPD peaking listing of Compound 1, crystalline Form III is provided in Table 3.

TABLE 3

| 2-Theta (°) | Relative Intensity (%) |
|---|---|
| 7.8 | 3 |
| 11.6 | 3 |
| 13.0 | 100 |
| 13.3 | 11 |
| 14.2 | 8 |
| 15.8 | 6 |
| 16.3 | 2 |
| 16.8 | 37 |
| 18.9 | 3 |
| 19.2 | 5 |
| 19.5 | 5 |
| 21.4 | 5 |
| 21.8 | 16 |
| 24.4 | 5 |
| 25.6 | 25 |
| 25.8 | 38 |
| 26.1 | 17 |
| 26.7 | 6 |
| 27.1 | 11 |
| 28.0 | 25 |
| 28.8 | 9 |
| 30.6 | 6 |
| 31.7 | 7 |
| 32.4 | 4 |

The DSC thermogram indicated a small endothermic transition at about 125° C. attributed to a form change from Compound 1, crystalline Form III to Compound 1, crystalline Form V, as shown in FIG. 9 (confirmed by XRPD in another experiment). Further heating the sample (at 150° C.) in another experiment resulted in a mixture of Compound 1, crystalline Form V and Compound 1, crystalline Form I, which was indicated by the double peaks in the temperature range from 150 to 165° C. The TGA thermogram showed no substantial weight loss (0.04% from 25° C. to 150° C.), indicating very low residual solvent, as shown in FIG. 10. The DVS analysis is shown in FIG. 11 and indicated that the form is slightly hygroscopic showing around a 1.2% weight change from 0 to 90% RH at 25° C.

### Example 3. Preparation of Compound 1, Crystalline Form IV

To a 4-mL vial equipped with a Teflon coated magnetic stir bar, about 50 mg of Compound 1, free base crystalline Form I was stirred with about 0.5 mL toluene at about 22° C. After three days, XRPD analysis of the wet solids showed a new pattern, designated as Compound 1, toluene solvate (see FIG. 12). After drying at 50° C. under vacuum, the XRPD pattern changed and the new form was designated as Compound 1, crystalline Form IV.

A representative XRPD pattern for Compound 1, crystalline Form IV is shown in FIG. 13 and is characterized by sharp reflections, indicating crystallinity. An XRPD peaking listing of Compound 1, crystalline Form IV is provided in Table 4.

TABLE 4

| 2-Theta (°) | Relative Intensity (%) |
|---|---|
| 8.4 | 6 |
| 12.2 | 20 |
| 12.8 | 69 |
| 13.3 | 9 |
| 14.8 | 23 |
| 15.9 | 15 |
| 16.3 | 11 |
| 16.8 | 3 |
| 17.6 | 5 |
| 18.0 | 20 |
| 20.3 | 4 |
| 24.4 | 47 |
| 24.8 | 100 |
| 25.8 | 18 |
| 26.5 | 3 |
| 26.9 | 3 |
| 27.9 | 4 |
| 29.4 | 4 |

The DSC thermogram indicated a small endothermic transition at about 114° C. attributed to a form change from Compound 1, crystalline Form IV to Compound 1, crystalline Form I, as shown in FIG. 14 (confirmed by XRPD in another experiment). The TGA thermogram showed no substantial weight loss (0.004% from 25 to 150° C.) indicating very low residual solvent, as shown in FIG. 15. The DVS analysis is shown in FIG. 16 and indicated that the form is non-hygroscopic showing around a 0.16% weight change from 0 to 90% RH at 25° C.

### Example 4. Preparation of Compound 1, Crystalline Form V

Compound 1, crystalline Form V can be prepared via any of the following procedures:
1. Heating Compound 1, crystalline Form II to about 100° C.
2. Heating Compound 1, crystalline Form III to about 125° C. Since Compound 1, crystalline Form III may be obtained by drying different solvates, the temperature required to convert Compound 1, crystalline Form III to Compound 1, crystalline Form V may vary.
3. Drying Compound 1, xylene solvate or DMAc solvate (see FIG. 17) at 75-110° C. A small amount of Compound 1, crystalline form Form I may also be present.

A representative XRPD pattern for Compound 1, crystalline Form V is shown in FIG. 18 and is characterized by sharp reflections, indicating crystallinity. An XRPD peaking listing of Compound 1, crystalline Form V is provided in Table 5.

TABLE 5

| 2-Theta (°) | Relative Intensity (%) |
|---|---|
| 5.4 | 14 |
| 9.0 | 32 |
| 10.8 | 6 |
| 11.2 | 42 |
| 11.9 | 12 |
| 13.9 | 4 |
| 14.4 | 8 |
| 15.1 | 68 |
| 15.4 | 30 |
| 16.7 | 15 |
| 17.3 | 12 |
| 18.0 | 61 |
| 19.6 | 100 |
| 20.3 | 5 |
| 20.9 | 81 |
| 22.3 | 24 |
| 23.8 | 9 |
| 24.0 | 8 |
| 24.9 | 22 |
| 27.2 | 19 |
| 28.6 | 11 |
| 29.3 | 12 |
| 30.5 | 6 |

The DSC thermogram indicated a melting onset at about 156° C., as shown in FIG. 19. The TGA thermogram showed no substantial weight loss (0.12% from 25 to 150° C.) indicating very low residual solvent, as shown in FIG. 20. The DVS analysis is shown in FIG. 21 and indicated that the form is non-hygroscopic showing around a 0.1% weight change from 0 to 90% RH at 25° C.

### Example 5. Summary of Compound 1, Solid Forms

The melting onset and DVS properties of Compound 1, crystalline Forms I-V are shown in Table 6.

TABLE 6

| | | DVS |
|---|---|---|
| Form | Melting Onset | (%) |
| I | 158° C. | 0.85 |
| II | Converts to Form V at about 100° C. | 0.08 |
| III | Converts to Form V at about 125° C. | 1.2 |
| IV | Converts to Form V at about 117.5° C. | 0.16 |
| V | 156° C.; May partially convert to | 0.10 |
| | Form I at about 145° C. to 150° C. | |

The DVS values of Forms II, III, IV, and V demonstrate the unique hygroscopicity properties of these novel forms. These properties can impart unexpected advantages such as improving the manufacturing process of the compound, increasing the stability or storability of a drug product form of the compound, increasing the stability or storability of a drug substance of the compound, improving the bioavailability and/or stability of the compound as an active agent, and increasing the physical and/or chemical stability of the dosage or delivery form.

All references, including publications, patents, and patent documents are incorporated by reference herein, as though individually incorporated by reference. The present disclosure provides reference to various embodiments and techniques. However, it should be understood that many variations and modifications may be made while remaining within the spirit and scope of the present disclosure.

1. A crystalline form of (2R,3S,5R)-5-(6-amino-2-fluoro-9H-purin-9-yl)-2-ethynyl-2-((2-phenylacetoxy)methyl)tetrahydrofuran-3-yl 2-phenylacetate, which is selected from crystalline Form II, crystalline Form III, crystalline Form IV, and crystalline Form V.

2. The crystalline form of claim 1, which is crystalline Form II.

3. The crystalline form of claim 2, wherein the crystalline Form II has at least three XRPD peaks, in terms of 2-theta±0.2°, selected from 5.5°, 9.3°, 10.8°, 14.9°, 18.5°, 19.3°, 23.8°, 24.3°, and 28.4°.

4. (canceled)

5. The crystalline form of claim 2, wherein the crystalline Form II is characterized by a DSC thermogram having an endothermic transition at about 93° C.

6. (canceled)

7. The crystalline form of claim 1, which is crystalline Form III.

8. The crystalline form of claim 7, wherein the crystalline Form III has at least three XRPD peaks, in terms of 2-theta±0.2°, selected from 7.8°, 11.6°, 13.0°, 14.2°, 16.8°, 21.8°, 25.8°, 26.1°, and 28.0°.

9. (canceled)

10. The crystalline form of claim 7, wherein the crystalline Form III is characterized by a DSC thermogram having an endothermic transition at about 125° C.

11. (canceled)

12. The crystalline form of claim 1, which is crystalline Form IV.

13. The crystalline form of claim 12, wherein the crystalline Form IV has at least three XRPD peaks, in terms of 2-theta±0.2°, selected from 8.4°, 12.2°, 12.8°, 14.8°, 15.9°, 18.0°, 24.4°, 24.8°, and 25.8°.

14. (canceled)

15. The crystalline form of claim 12, wherein the crystalline Form IV is characterized by a DSC thermogram having an endothermic transition at about 114° C.

16. (canceled)

17. The crystalline form of claim 1, which is crystalline Form V.

18. The crystalline form of claim 17, wherein the crystalline Form V has at least three XRPD peaks, in terms of 2-theta±0.2°, selected from 5.4°, 9.0°, 11.2°, 15.1°, 15.4°, 18.0°, 19.6°, 20.9°, and 22.3°.

19. (canceled)

20. The crystalline form of claim 17, wherein the crystalline Form V is characterized by a DSC thermogram having a melting onset of about 156° C.

21. (canceled)

22. A solvate form of (2R,3S,5R)-5-(6-amino-2-fluoro-9H-purin-9-yl)-2-ethynyl-2-((2-phenylacetoxy)methyl)tetrahydrofuran-3-yl 2-phenylacetate.

23. The solvate form of claim 22, which is selected from, an acetone solvate form, a methyl ethyl ketone solvate form, a dichloromethane solvate form, a tetrahydrofuran solvate form, a toluene solvate form, a n-butyl acetate solvate form, a methyl acetate solvate form, a xylene solvate form, a heptane solvate form, a 1-butanol solvate form, a p-dioxane solvate form, a DMAc solvate form, an ethyl acetate solvate form, and a dimethylacetamide solvate form.

24.-42. (canceled)

43. A pharmaceutical composition comprising the crystalline form of claim 1, and at least one pharmaceutically acceptable excipient.

44. A method of treating or preventing a human immunodeficiency virus (HIV) infection comprising administering a therapeutically effective amount of the crystalline form of claim 1, to a subject in need thereof.

45.-46. (canceled)

47. A pharmaceutical composition comprising the solvate form of claim 22, and at least one pharmaceutically acceptable excipient.

48. A method of treating or preventing a human immunodeficiency virus (HIV) infection comprising administering a therapeutically effective amount of the solvate form of claim 22, to a subject in need thereof.
